(19) United States
(12) Patent Application Publication
(10) Pub. No.: US 2006/0286683 A1
(43) Pub. Date: Dec. 21, 2006

(54) Method and testing system for analyzing and/or detecting biomolecules and/or active substances in liquid samples

(75) Inventors: Gerhard Hermann, Wuerzburg (DE); Ilona Kuehlmann-Rabens, Creglingen (DE); Uwe Schobel, Wetzlar (DE)

Correspondence Address: JOYCE VON NATZMER;Hall, Vande Sande & Pequigot, LLP, 10220 River Road, Suite 200, Potomac, MD, 20854, US

(73) Assignee: Insitut Virion\Serion GmbH, Wuerzsburg (DE)

(21) Appl. No.: 10/558,198

(22) PCT Filed: May 21, 2004
(86) PCT No.: PCT/DE04/01078

§ 371 Date: Nov. 25, 2005

(30) Foreign Application Priority Data
May 26, 2003 (DE) 103239014

(51) Int. Cl. G01N 33/551

(52) U.S. Cl. 436/524; 977/900

(57) ABSTRACT

The method for assaying and/or detecting the type and/or quantity of at least one sample substance is characterized by the following features: pre-dosed quantities of carrier particles, which differ in regard to size and/or color (type of color and/or color intensity), are loaded with probe/detector molecules, and are dried, are provided. These carrier particles are either (i) brought into contact with the liquid which contains the sample substance(s) to be assayed and/or detected, the sample substance(s) being marked with one and/or more defined marking variables, or they are (ii) brought into contact simultaneously or sequentially with sample substance(s) and with marker/reporter substance(s), which are located in the same or in different liquids. After one or more reaction times, the size and/or color of the carrier particles and at least one marking variable of the sample substance(s) and/or marker/reporter substance(s) are analyzed, from which the type and/or quantity of the sample substance(s) may be concluded. The test system is characterized in that it comprises groups, which differ in regard to size and/or color, of carrier particles loaded with probe and/or detector molecules, which are provided pre-dosed in dried form.

The present invention relates to a method for assaying and/or detecting the type and/or quantity of at least one sample substance to be assayed, in which (a) pre-dosed quantities of carrier particles are provided, which are loaded with probe and/or detector molecules, (b) these loaded carrier particles are provided in dried form, (c) (i) the carrier particles are brought into contact with a liquid which contains the sample substance(s) to be assayed and/or detected, the sample substance(s) being marked with one or more defined marking variables, or (ii) the carrier particles are brought into contact simultaneously or sequentially with the sample substance(s) and with marker and/or reporter substance(s), which are located in the same or different liquids, and (d) after one or more reaction times, during which the sample substance(s) and probe and/or detector molecules and marker and/or reporter substance which have been brought into contact may bind, at least one (but preferably two or more) defined marking variables are analyzed, from which the quantity of -the sample substance(s) to be assayed and/or detected may be concluded.

A method is described in DE 33 22 373 C2 and EP 0 126 450 which allows the simultaneous determination of multiple antigens and/or antibodies from a sample. A mixture made of particles which are coated with different antibodies and/or antigens is mixed with a liquid which contains the antigens and/or antibodies to be assayed and/or detected. After a defined reaction time, in which the antigens and/or antibodies to be assayed are bound by the antibodies and/or antigens coated on the carrier particles, the bound antigens and/or antibodies are identified by adding a liquid having fluorescence-marked antibodies and/or antigens, which react species-specifically with the antigens and/or antibodies to be detected and/or assayed. The analysis of the markings of the individual carrier particles is performed with the aid of special flow cytometers tailored for particle measurements. The composition and/or the presence of antigens and/or antibodies to be assayed may be concluded from this analysis. Through suitable selection of the antibodies and/or antigens, with which the carrier particles used are loaded, predefined desired properties of the liquid to be assayed and/or its contents may be assayed. Depending on which antigens and/or antibodies are to be detected and/or analyzed in the liquid to be assayed, test particles which are loaded with appropriate corresponding antibodies and/or antigens may be used.

In the present text, “sample substance(s)” refers to the biomolecules and/or active ingredients to be assayed and/or detected. In this case, the term “biomolecules” stands for all molecules and molecular fragments which occur naturally in animate and inanimate nature and may be provided either through isolation methods or identical replication, particularly molecules in/or from plant and animal cells, organs, and organisms, in/from prokaryotes, in/from fungi, in/from viruses and phages, but also molecules such as virions; prions, and similar things or parts thereof (e.g., antigens, antibodies, DNA, DNA fragments, etc.).

The term “active ingredients” stands here for all molecules which have been created and synthesized by human hand (e.g., synthetic medications).

“Probe molecules and/or detector molecules” refers to those biomolecules and/or active ingredients, with which the carrier particles are loaded and/or coated, i.e., which are located on/in these carrier molecules.

“Marker and/or reporter substance” refers to those biomolecules and/or active ingredients which have a known, defined marking variable, which is used for analysis, i.e., is evaluated. In particular, biomolecules or active ingredients, which have fluorescence activity, phosphorescence activity, bioluminescence activity, chemiluminescence, chromophore activity, radioactivity, or enzyme activity or are coupled to molecules which have one or more of these activities, come into consideration as such marker and/or reporter substances.

The method known in the related art comprises the following steps:
dispensing the mixture made of particles which are coated with second antibodies/antigens into a reaction vessel as the probe/detector molecules,
adding a liquid which contains the first antigens/antibodies to be assayed and/or detected as the sample substance(s),
incubation,
washing the carrier particles after the end of the reaction time to remove the non-bound substances and molecules,
adding a liquid having fluorescence-marked third antibodies and/or antigens as marker and/or reporter substance(s), which bind with the first antigens/antibodies to be detected and/or assayed (i.e., the sample substance(s)) species-specifically,
incubation,
washing to remove the non-bound substances and molecules, and
analyzing the markings of the individual carrier particles with the aid of special flow cytometers tailored for carrier particle measurements.

At the beginning of the analysis method, the carrier particles and/or particle mixture, which are coated with the desired biomolecules/active ingredients as the probe and/or detector molecules, are to be dispensed into a reaction vessel. This pipetting step influences the quality of the assaying results very decisively, since the concentration of the sample and/or detector molecules located on the particles in the test solution is determined in this way. The concentration of the biomolecules/active ingredients located on the particles in the test solution has an influence on the test setting and the test level and therefore on the evaluation of the assayed sample material. Particle counts deviating from the test setting also have an influence on the count rates, e.g., during the analysis in a flow cytometer.

If the carrier particles to be dispensed are provided, for example, in aqueous buffer solutions (such as PBS), a sedimentation process occurs because of the higher density of the carrier particles (e.g., 1.05 g/ml for carrier particles made of polystyrene). Before further use, the suspension must thus be homogenized, foaming of the suspension and the formation of air bubbles having to be carefully avoided. Care must be taken in the following process that renewed sedimentation of the carrier particles is avoided.

The present invention is based on the object of refining the known method in such a way that the cited disadvantages are avoided and simplified method control is possible with high sensitivity and specificity at the same time.

This object is achieved by a method of the type cited at the beginning which is distinguished in that the carrier particles loaded with the sample and/or detector molecules differ in regard to size and/or color, and the size and/or color of the carrier particles is also analyzed in step (d).

“Differ in regard to color” means, in the present context, that the carrier particles are and/or may be provided (coded) with different types of colors and/or coloring agents (e.g., red, green, blue) and also with different color intensities of the same type of color/the same coloring agents (e.g., in ten intensity stages from bright red to dark red). Therefore, the type of color and/or the color intensity is analyzed during the analysis of the color.

In the method according to the present invention, the carrier particles loaded and/or coated with the probe and/or detector molecules are provided pre-dosed in dried form. A laboratory worker may thus make use of the pre-dosed quantity of the coated carrier particles without having to first precisely determine the quantity of the carrier particles in a complex pipetting step. During the assaying and/or the detection of the sample substance(s), this especially critical and error-intensive step is thus no longer necessary and the method may thus also be performed under simplified conditions without great expertise. The step of precisely determining the quantity of the carrier particles is performed beforehand when manufacturing corresponding test systems.

Since the pre-dosed quantity of carrier particles is provided in dried form, preferably in freeze-dried form, extended storability of the probe and/or detector molecules with which the carrier particles are coated is additionally provided.

Providing the carrier particles in freeze-dried form is especially simple to produce and precise.

The method according to the present invention may be used both for assaying the quantity of a specific existing sample substance (i.e., types of biomolecules and/or active ingredients) and also for detecting whether a specific sample substance (i.e., corresponding biomolecules or active ingredients) is contained at all.

In a preferred embodiment variation, carrier particles are used for this purpose which differ not only in color and/or size, but rather are additionally loaded with different probe and/or detector molecules.

The pre-dosed quantity of carrier particles having probe and/or detector molecules located thereon may particularly comprise multiple groups of carrier particles for this purpose, the individual groups differing from one another firstly through the size and/or coloration (type of color and/or color intensity) of the associated carrier particles and secondly through the type of the probe and/or detector molecules on the carrier particles. In other words, the carrier particles of each individual group have the same size and type of color/color intensity and are loaded with the same probe and/or detector molecules, the carrier molecules of second groups differ in their size and/or their type of color and/or color intensity and they are loaded with different probe and/or detector molecules. Using this embodiment variation of the method according to the present invention, detection and/or assaying of different sample substances in the same test liquid may be performed extremely easily in one simple assaying procedure.

For this purpose, probe and/or detector molecules which are complementary to the sample substance(s) and/or to the marker and/or reporter substance(s) may be used. In this case, probe and/or detector molecules (i.e., biomolecules or active ingredients) which bind further sample substance(s) (biomolecules and/or active ingredients) via an affinity and/or hybridization reaction and/or covalent reaction are referred to as complementary.

In order to test whether a specific type of sample molecule (i.e., biomolecule and/or active ingredient) is contained in a liquid sample at all, carrier particles having sample and/or detector molecules which (would) react with the sample substance possibly to be detected are preferably used.

The method according to the present invention may also be performed while exploiting a competitive reaction. For this purpose, the sample substance(s) and the probe and/or detector molecules are very similar or even identical and the marker and/or reporter substances are complementary to the sample substances and/or the probe and/or detector molecules. During the competitive reaction, the sample substance(s) compete with the probe and/or detector molecules for binding to the marked complementary marker and/or reporter substance. If no sample substance molecules are contained in a liquid, all marker and/or reporter substance molecules can bind to the probe/detector molecules and therefore to the carrier particles and are detected. However, if many sample substance molecules are present, all marker and/or reporter substance molecules bind to the sample substance molecules and not to the probe/detector molecules and therefore not to the carrier particles and are therefore not detected.

In an advantageous embodiment of the method according to the present invention, the pre-dosed and dried and/or freeze-dried carrier particles are provided as pressed parts (pellets). In other words, pressed parts (pellets) are used which contain the dried and/or freeze-dried carrier particles in pre-dosed form. These pressed parts (pellets) are easy to handle and may be used in any arbitrary reaction vessels.

A refinement of this method variation provides that different groups of pressed parts (pellets) are used which differ through the differing load of the carrier particles contained therein with probe and/or detector molecules, the carrier particles of each individual pressed part (pellets) being loaded with the same probe and/or detector molecules.

For example, for an infection serology test during pregnancy (“pregnancy panel”), a single pressed part (pellet) may be provided which contains different size and/or different color carrier particles, which are loaded with probe and/or detector molecules for the antigens and/or antibodies of rubella, toxoplasmosis, CMV, HSV, VZV, parvovirus, etc., each carrier particle species being loaded with a specific probe and/or detector molecule species.

For example, a set of multiple pressed parts (pellets) may be provided equally well for an infection serology test for vaccination control, each pressed part (pellet) only containing one single specific group of carrier particles loaded with a specific type of probe and/or detector molecule for the antigen and/or antibody of tetanus, diphtheria, pertussis, etc., for example. These individual pressed parts (pellets) may then be mixed together individually depending on the question.

The pre-dosed, dried and/or freeze-dried carrier particles may just as well be provided unpressed in reaction vessels such as microtitration plates or test tubes, and these reaction vessels may then be used directly for analysis of the liquid to be assayed without further additional measures. The laboratory worker must only pour the liquid to be assayed into this reaction vessel in order to bring it into contact with the pre-dosed and therefore known quantity of freeze-dried carrier particles, a transferring process or a quantity determination is no longer necessary.

A fluorescent dye (molecule) is suggested as the defined marking variable for the sample substance(s) in the liquid, so that in step (d) of the method according to the present invention, the fluorescence is therefore analyzed.

The method according to the present invention may be simplified especially advantageously if the pre-dosed carrier particles, which are loaded with probe and/or detector molecules, are provided together with a pre-dosed quantity of marker and/or reporter substance(s) in dried and/or freeze-dried form. For this purpose, the marker and/or reporter substance(s) has/have the property of reacting species-specifically with the sample substance(s) to be assayed and/or detected. It/they may comprise one or more components and it/they may have one or more structures complementary to the probe and/or detector molecules and/or sample substance(s).

In such a method, in which marked marker and/or reporter substance(s) are already provided with the pre-dosed carrier particles, further reduction of the error rate is ensured by saving a further dispensing step. In addition, the procedure is further simplified through reduction of the number of method steps, because in contrast to the method steps of the related art listed above, in this variation of the method according to the present invention, only adding a liquid which possibly contains the sample substance(s) to be assayed and/or detected and analyzing the markings of the individual carrier particles with the aid of special flow cytometers tailored for particle measurements, for example, are necessary.

The object of the present invention is also a test system (kit) for use in the assay and/or detection method according to the present invention, which is distinguished in that it comprises groups of carrier particles loaded with probe and/or detector molecules, which differ in regard to size and/or color (type of color and/or color intensity), and which are provided pre-dosed in dried form. The carrier particles are preferably coded in regard to the color in a known way by introducing one or more fluorophores, possibly in different concentrations, into the carrier particles or by attaching one or more identical or different fluorescing nanoparticles to the surface of the carrier particles.

This test system (kit) according to the present invention may be used directly by a laboratory worker without a pipetting or dispensing step, so that a significant source of errors is reduced in the way described. Such test systems (kits) may be provided in large quantities beforehand and, for example, sold as such.

The carrier particles are preferably provided in the test system (kit) in freeze-dried form.

The test system may comprise groups of carrier particles which differ in regard to color and/or size, the carrier particles of each group being loaded with identical probe and/or detector molecules and the carrier particles of different groups carrying different probe and/or detector molecules.

The dried and/or freeze-dried carrier particles are preferably provided in the test system either pre-dosed in the form of one or more pressed part(s) (pellet/pellets) or pre-dosed unpressed in a reaction vessel.

In an embodiment variation of the test system according to the present invention, it additionally comprises at least one marker and/or reporter substance which may react species-specifically with the sample substance(s) to be assayed. This marker and/or reporter substance may particularly have a fluorescence activity, phosphorescence activity, bioluminescence activity, chemiluminescence activity, chromophore activity, radioactivity, or enzyme activity.

The marker and/or reporter substance(s) is/are preferably either contained in the pressed parts (pellets) or in the reaction vessels filled with the carrier particles and may comprise one or more components.

The advantages already described above for the method according to the present invention apply analogously for these special embodiments of the test system (kit).

All test systems (kits) according to the present invention are preferably manufactured at a central location and sold from there, so that they may be used directly for the desired assays in the laboratory after acquisition without additional dispensing or pipetting steps.

The method according to the present invention and the test system according to the present invention will be explained in greater detail in the following on the basis of figures and an exemplary embodiment. In this case, it is obvious that the procedure described in the examples for the relevant assay also applies in principle for any other assay of a liquid sample in regard to sample substance(s) located therein.

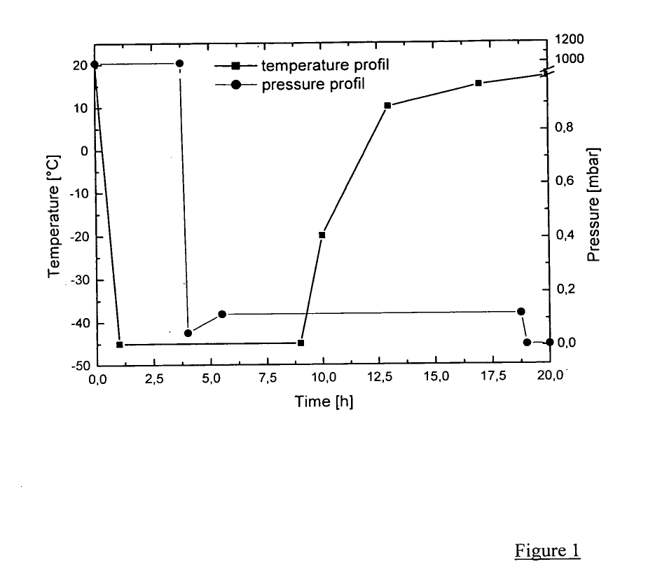

FIG. 1 shows an example (other temperature and pressure profiles are also conceivable, of course) of the temperature and/or pressure curve during the freeze drying when performing a manufacturing method according to the present invention for the test systems (kits) according to the present invention.

### EXAMPLE 1

### Infection Serology Assay for Pregnant Women—Variation 1

In order to obtain information about the presence and/or the composition of specific sample substance(s), such as antigens and/or antibodies of rubella, toxoplasmosis, CMV, HSV, VZV, and parvovirus in the serum sample of a pregnant woman, different sized carrier particles are loaded with different probe and/or detector molecules, specifically probe and/or detector molecules for these and possibly further antigens and/or antibodies, pre-dosed, freeze-dried, and provided—either unpressed in a reaction vessel or compressed into a pressed part (pellet).

To analyze this serum sample, a laboratory worker makes use of such a reaction vessel or such a pressed part (pellet) having a pre-dosed quantity of carrier particles. He knows how many carrier particles, and therefore probe and/or detector molecules, he is using, and for which of the sample substance(s) to be assayed and/or possibly detected the probe and/or detector molecules are specific. The laboratory worker brings the pressed part (pellet) or the unpressed quantity of carrier particles in a reaction vessel into contact with the liquid to be assayed, which contains the sample substance(s), specifically with the serum sample, in a and/or the reaction vessel. Simultaneously or sequentially, he adds a specific quantity of marker and/or reporter substance(s).

This step is superfluous if the marker and/or reporter substance(s) is/are already provided together with the carrier particles loaded with the probe and/or detector molecules dried, pre-dosed, and either compressed into a pressed part (pellet) or poured unpressed into a reaction vessel.

After expiration of the reaction time, different sample substance(s) and identical marker and/or reporter substance(s) are bound to the carrier particles. By analyzing the size of the carrier particles—e.g., in a flow cytometer tailored for particle measurements—the type and, through analysis of the defined marking variable(s) of the sample substance(s) and/or marker and/or reporter substance(s), the quantity of sample substance(s) in the liquid to be assayed may be concluded.

To remove unbound substances, washing steps known per se may be performed at the end of the particular reaction times.

### EXAMPLE 2

### Infection Serology Assay for Pregnant Women—Variation 2

As in Example 1, however, instead of the different sized carrier particles, different colored carrier particles, specifically carrier particles which differ in regard to the type of color and/or color intensity, are used and loaded with the different probe and/or detector molecules. By analyzing the different colors and/or color intensities, the different carrier particles and therefore the type of probe and/or detector molecules bound thereto and the type of the sample substance(s) detected therewith may be detected, and by analyzing the defined marking variable (s) of the sample substance(s) and/or the marker and/or reporter substance(s), conclusions may be drawn about the quantity of the sample substance(s) in the liquid to be assayed.

### EXAMPLE 3

### Infection Serology Assay for Pregnant Women—Variation 3

As in Example 2, however, carrier particles are used which are different sized and different colored, the differences in color being able to be a different type of color or a different color intensity (with the same type of color) Carrier particles of the same size and type of color or color intensity, i.e., the same coding, are loaded with probe and/or detector molecules of the same type, carrier particles of a different size and/or type of color and/or color intensity, i.e., having a different coding, are loaded with a different type of probe and/or detector molecules.

By analyzing the different size and/or type of color and/or color intensity, the different carrier particles and therefore the type of probe and/or detector molecules bound thereto and the type of the sample substance(s) detected therewith are detectable, and by analyzing the defined marking variable (s) of the sample substance(s) and/or the marker and/or reporter substance(s), conclusions may be drawn about the quantity of the sample substance(s) in the liquid to be assayed.

### EXAMPLE 4

### Detection of Sample Substance(s) Without using Marker and/or Reporter Substance(s)

To detect nucleic acid sequences (e.g., DNA), the sample substance(s), e.g., a specific DNA, may already be fluorescence marked. For such a test, the DNA to be detected is first amplified. This step may be performed using primers which are already fluorescence marked, so that the amplificates of the DNA to be detected (i.e., the sample substance) already contain a fluorophore.

Probe and/or detector molecules, e.g., in the form of a second (complementary) DNA are coupled to the carrier particles, which are coded in regard to size and color (type of color and/or color intensity). The fluorescence-marked amplificates of the DNA to be detected (sample substance(s)) bind directly to the (second) probe and/or detector DNA on/in the carrier particles.

Using marker and/or reporter substance(s) is not necessary. Therefore, a test system according to Example 5 is suggested for performing this method.

However, there is also the possibility of using marked and non-marked third DNA molecules as the marker and/or reporter substances, which function as a bridge between the sample DNA and the probe and/or detector DNA. A test system according to Example 6 is suitable for performing this method, specifically a test system which comprises carrier particles loaded with probe and/or detector DNA and marked or non-marked marker and/or reporter DNA in dried, pre-dosed form.

### EXAMPLE 5

### Manufacture and Composition of a Test System (Kit) According to the Present Invention—Variation 1

One or more different types of carrier particles (i.e., carrier particles having the same or different codings) are dispensed in defined quantities and under monitored conditions into reaction vessels, a special buffer stabilizer system (e.g., 50 mM phosphate buffer, 150 mM sodium chloride, 0.02% sodium azide, 8 g/L gelatin hydrolysate, pH 7.4, and additionally skeletal substances and auxiliary materials such as sucrose, phenylalanine) preferably being used. If necessary, the carrier particles are homogenized using ultrasound and/or mechanical stirring. Subsequently, these pre-dosed carrier particles are dried using freeze drying or other drying methods and are thus made storable and simpler to handle. For this purpose, a temperature and pressure profile is used as may be inferred from FIG. 1. Depending on which analysis the test system is to be used for, either (a) carrier particles of the same coding and loaded with only one type of probe and/or detector molecule or (b) carrier particles of the same coding but loaded with different types of probe and/or detector molecules, or (c) carrier particles of different codings and loaded with only one type of probe and/or detector molecules per coding are used.

After the drying/freeze drying, these particles loaded with probe and/or detector molecules may be molded into pressed parts (pellets), these pressed parts (pellets) each containing a predefined quantity of carrier particles.

In the case of pressed parts (pellets) which only contain one type of carrier particles having a specific size and specific type of color and/or color intensity, i.e., only containing carrier particles of the same coding, and these carrier particles being loaded with only one type of complementary probe and/or detector molecules, the mixture of the carrier particles may be produced individually by combining corresponding pressed parts (pellets) in a reaction vessel.

### EXAMPLE 6

### Manufacture and Composition of a Test System (Kit) According to the Present Invention—Variation 2

As in Example 5, however, the carrier particles loaded with the probe and/or detector molecules are dispensed into the reaction vessels together with a defined quantity of marker and/or reporter substance(s) using a special buffer-stabilizer system under precisely monitored conditions, dried using freeze drying or other drying methods, and finally provided compressed into pressed parts (pellets) or poured unpressed into reaction vessels.

1. A method for assaying and/or detecting the type and/or quantity of at least one sample substance comprising:
a) providing pre-dosed quantities of carrier particles which are loaded with probe and/or detector molecules,
b) wherein these loaded carrier particles are provided in dried form,
c) (i) bringing the carrier particles into contact with a liquid which contains the sample substance(s) to be assayed and/or detected, the sample substance(s) being marked with one and/or more defined marking variables, or (ii) bringing the carrier particles into contact simultaneously or sequentially with sample substance(s) and marker and/or reporter substance(s), which are located in the same or in different liquids, and
d) analyzing after one or more reaction times, during which the sample substance(s) and probe and/or detector molecules and marker and/or reporter substance(s) brought into contact with one another may bind, at least one marking variable is from which the type and/or quantity of the sample substance(s) may be concluded,
wherein the carrier particles loaded with the probe and/or detector molecules differ in regard to size and/or color (type of color and/or color intensity), and
wherein the size and/or color of the carrier particles is also analyzed in step (d).

2. The method according to claim 1,
wherein the carrier particles are freeze-dried.

3. The method according to claim 1, wherein the carrier particles, which differ according to color and/or size, are loaded with different probe and/or detector molecules.

4. The method according to claim 2, wherein the dried and/or freeze-dried carrier particles are provided as pressed parts (pellets).

5. The method according to claim 4, wherein different groups of pressed parts (pellets) are used, which differ through the differing load of the carrier particles contained therein with probe and/or detector molecules, the carrier particles of each individual pressed part (pellet) being loaded with the same probe and/or detector molecules.

6. The method according to claim 1, wherein the dried carrier particles are provided pre-dosed in reaction vessels.

7. The method according to claim 1, wherein the defined marking variable of the sample substance(s) is a fluorescent dye and the fluorescence is analyzed in step d).

8. The method according to claim 1, wherein the marker and/or reporter substance has fluorescence activity, phosphorescence activity, bioluminescence activity, chemiluminescence, chromophore activity, radioactivity, or enzyme activity.

9. The method according to claim 1, wherein the pre-dosed carrier particles, which are loaded with probe and/or detector molecules, are provided in dried and/or freeze-dried form together with a pre-dosed quantity of marker and/or reporter substance(s), which is/are capable of reacting species-specifically with the sample substance(s) to be assayed and/or detected.

10. A test system for assaying and/or detecting the type and/or quantity of at least one sample substance comprising
a) groups of carrier particles loaded with probe and/or detector molecules,
b) wherein these loaded carrier particles are predosed in dried form and differ in regard to size and/or color (type of color and/or color intensity),
c) (i) a structure to bring the carrier particles into contact with a liquid which contains the sample substance(s) to be assayed and/or detected, the sample substance(s) being marked with one and/or more defined marking variables, or (ii) a structure to bring the carrier particles into contact simultaneously or sequentially with sample substance(s) and marker and/or reporter substance(s), which are located in the same or in different liquids, and
d) analyzer for analyzing, after one or more reaction times, during which the sample substance(s) and probe and/or detector molecules and marker and/or reporter substance(s) brought into contact with one another may bind, at least one marking variable from which the type and/or quantity of the sample substance(s) may be concluded, and
wherein the analyzer of (d) also analyzes the size and/or color of the carrier particles.

11. The test system according to claim 10, wherein the carrier particles of the same group are loaded with identical probe and/or detector molecules and the carrier particles of different groups carry different probe and/or detector molecules.

12. The test system according to claim 10, wherein the carrier particles are provided freeze-dried.

13. The test system according to claim 10, wherein the dried carrier particles are provided pre-dosed in the form of one or more pressed part(s) (pellet/pellets).

14. The test system according to claim 10, wherein the dried carrier particles are provided pre-dosed in a reaction vessel.

15. The test system according to claim 10 further comprising at least one marker and/or reporter substance.

16. The test system according to claims 15, wherein the marker and/or reporter substance has fluorescence activity, phosphorescence activity, bioluminescence activity, chemiluminescence, chromophore activity, radioactivity, or enzyme activity.

17. The method according to claim 2, wherein the freeze-dried carrier particles are provided pre-dosed in reaction vessels.

18. A test system for use in a method according to claim 1 comprising groups of carrier particles loaded with probe and/or detector molecules, which differ in regard to size and/or color and are provided pre-dosed in dried form.
